(19) United States
(12) Patent Application Publication
(10) Pub. No.: US 2018/0146944 A1
(43) Pub. Date: May 31, 2018

(54) DYNAMIC IMAGE PROCESSING APPARATUS

(71) Applicant: Konica Minolta, Inc., Tokyo (JP)

(72) Inventor: Noritsugu MATSUTANI, Tokyo (JP)

(21) Appl. No.: 15/802,022

(22) Filed: Nov. 2, 2017

(30) Foreign Application Priority Data
Nov. 25, 2016 (JP) 2016-229486

(51) Int. Cl. A61B 6/00; G06T 7/00; G06T 5/10

(52) U.S. Cl. CPC A61B 6/5217; G06T 7/0012; G06T 5/10; G06T 2207/30048; A61B 6/563; G06T 2207/10116; G06T 2207/30061; A61B 6/503

(57) ABSTRACT

A dynamic image processing apparatus includes a hardware processor. The hardware processor obtains a frequency characteristic of density change of a dynamic image in a time direction. The dynamic image is obtained by imaging at least one breathing cycle or pulsation cycle. Further, the hardware processor obtains peak frequencies in a frequency range for a type of diagnosis target based on the obtained frequency characteristic. Further, the hardware processor determines at least one representative frequency including a maximum peak frequency an intensity of which is highest among the obtained peak frequencies. Further, the hardware processor performs time-direction filtering on the dynamic image using cutoff frequencies to emphasize the determined representative frequency.

### CROSS-REFERENCE TO RELATED APPLICATIONS

The present U.S. Patent Application claims a priority under the Paris Convention of Japanese Patent Application No. 2016-229486 filed on Nov. 25, 2016, the entire disclosure of which, including the description, claims, drawings and abstract, is incorporated herein by reference in its entirety.

### BACKGROUND

### 1. Technological Field

The present invention relates to a dynamic image processing apparatus.

### 2. Description of the Related Art

A dynamic image obtained by X-ray imaging of the dynamic state of a chest contains signal component(s) due to ventilation and signal component(s) due to lung perfusion, and the signal component due to lung perfusion is noise in diagnosis of ventilation whereas the signal component due to ventilation is noise in diagnosis of lung perfusion. Hence, there is described, for example, in Japanese Patent Application Publication No. 2014-128687 (Patent Document 1) a technique for extracting signal component(s) for the type of diagnosis target by performing time-direction frequency filtering on a dynamic image of a chest using cutoff frequencies based on whether the type of diagnosis target is ventilation or lung perfusion.

By the way, one of the objects of diagnosis of ventilation using a dynamic image of a chest is to identify local ventilation abnormal parts due to COPD, pulmonary emphysema or the like, and one of the objects of diagnosis of lung perfusion using a dynamic image of a chest is to identify local lung-perfusion abnormal parts due to acute pulmonary embolism, stenosis or the like. In order to achieve the above objects, readability of information in the dynamic image necessary for diagnosis is important. That is, it is necessary to extract the signal component due to ventilation or the signal component due to lung perfusion according to the object of diagnosis with high accuracy.

However, dynamic images contain, for example, electric white noise generated when signals are detected by a radiation detector. This white noise exists on all the frequency bands. Hence, filtering with the technique described in Patent Document 1 is not so effective in such noise. Further, breathing cycles and pulsation cycles depend on examinees, namely, there are individual differences in the cycles. The technique described in Patent Document 1 does not give consideration to the individual differences and hence sometimes cannot extract the signal component of the dynamic state to be diagnosed.

### SUMMARY

Objects of the present invention include enhancing readability of information on the dynamic state to be diagnosed in a dynamic image.

In order to achieve at least one of the abovementioned objects, according to an aspect of the present invention, there is provided a dynamic image processing apparatus including a hardware processor that: obtains a frequency characteristic of density change of a dynamic image in a time direction, the dynamic image being obtained by imaging at least one breathing cycle or pulsation cycle; obtains peak frequencies in a frequency range for a type of diagnosis target based on the obtained frequency characteristic; determines at least one representative frequency including a maximum peak frequency an intensity of which is highest among the obtained peak frequencies; and performs time-direction filtering on the dynamic image using cutoff frequencies to emphasize the determined representative frequency.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows the overall configuration of a dynamic image processing apparatus according to embodiments of the present invention;

FIG. 2 is a flowchart of an imaging control process performed by a controller of an imaging console shown in FIG. 1;

FIG. 3 is a flowchart of a frequency emphasis process A performed by a controller of a diagnostic console shown in FIG. 1;

FIG. 4 schematically shows procedure of frequency emphasis processes according to the embodiments;

FIG. 5 is a flowchart of a frequency emphasis process B performed by the controller of the diagnostic console shown in FIG. 1;

FIG. 6 is a flowchart of a frequency emphasis process C performed by the controller of the diagnostic console shown in FIG. 1; and

FIG. 7 is a flowchart of a frequency emphasis process D performed by the controller of the diagnostic console shown in FIG. 1.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

### [Configuration of Dynamic Image Processing System 100]

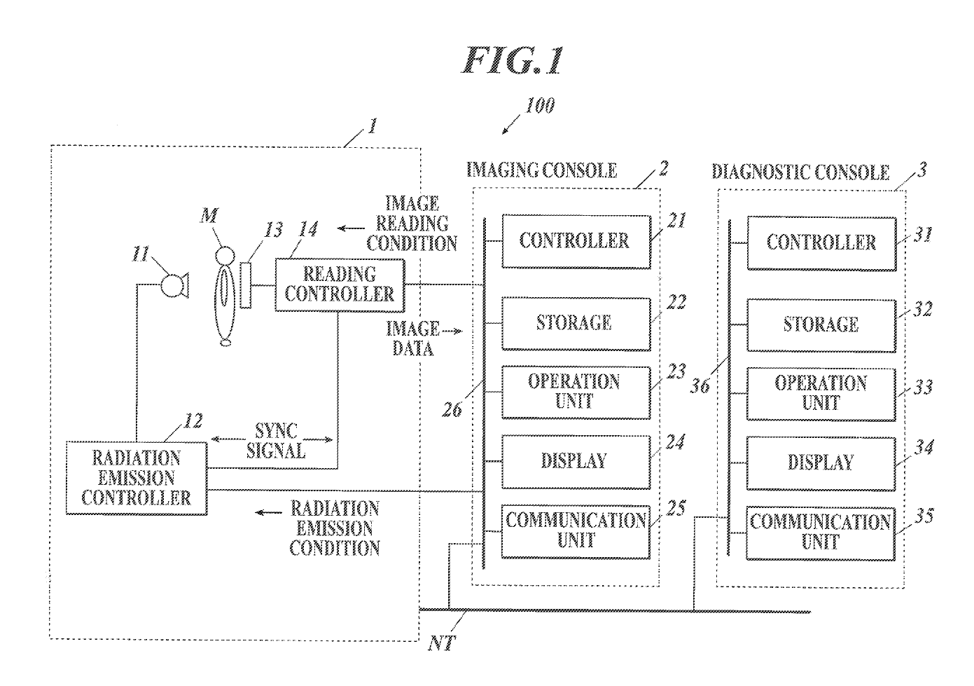

First, the configuration of a first embodiment is described. FIG. 1 shows the overall configuration of a dynamic image processing system 100 according to the embodiment(s) of the present invention.

As shown in FIG. 1, the dynamic image processing system 100 includes: an imager 1; an imaging console 2 connected with the imager 1 via a communication cable or the like; and a diagnostic console 3 connected with the imaging console 2 via a communication network NT, such as a LAN (Local Area Network). These apparatuses of the dynamic image processing system 100 are in conformity with DICOM (Digital Image and Communications in Medicine) standard and communicate with one another in conformity with DICOM.

### [Configuration of Imager 1]

The imager 1 is an imager that images a cyclic dynamic state of the chest as a subject, for example. Examples of the cyclic dynamic state thereof include: change in shape of the lungs by expansion and contraction of the lungs with breathing; and pulsation of the heart. Dynamic imaging (kinetic imaging) is performed by repeatedly emitting pulsed radiation, such as pulsed X-rays, to a subject at predetermined time intervals (pulse emission) or continuously emitting radiation without a break to a subject at a low dose rate (continuous emission), thereby obtaining a plurality of images. A series of images obtained by dynamic imaging is called a dynamic image Images constituting a dynamic image are called frame images. In the embodiments described hereinafter, dynamic imaging is performed by pulse emission as an example. Further, in the embodiments described hereinafter, a subject M is the chest of an examinee, but not limited thereto.

A radiation source 11 is disposed to face a radiation detector 13 with a subject M interposed therebetween, and emits radiation (X-rays) to the subject M under the control of a radiation emission controller 12.

The radiation emission controller 12 is connected with the imaging console 2, and controls the radiation source 11 on the basis of radiation emission conditions input from the imaging console 2 so as to perform radiation imaging The radiation emission conditions input from the imaging console 2 include a pulse rate, a pulse width, a pulse interval, the number of frames (frame images) to be taken by one imaging, a value of current of an X-ray tube, a value of voltage of the X-ray tube, and a type of added filter. The pulse rate is the number of times radiation is emitted per second, and matches the frame rate described below. The pulse width is a period of time for one radiation emission. The pulse interval is a period of time from the start of one radiation emission to the start of the next radiation emission, and matches the frame interval described below.

The radiation detector 13 is constituted of a semiconductor image sensor, such as an FPD. The FPD is constituted of detection elements (pixels) arranged at predetermined points on a substrate, such as a glass substrate, in a matrix. The detection elements detect radiation (intensity of radiation) that has been emitted from the radiation source 11 and passed through at least a subject M, convert the detected radiation into electric signals, and accumulate the electric signals therein. The pixels are provided with switches, such as TFTs (Thin Film Transistors). There are an indirect conversion type FPD that converts X-rays into electric signals with photoelectric conversion element(s) via scintillator(s) and a direct conversion type FPD that directly converts X-rays into electric signals. Either of them can be used. In the embodiments, pixel values (signal values) of image data generated in the radiation detector 13 are density values, and the larger the radiation passing amount is, the higher the pixel values are.

The radiation detector 13 is disposed to face the radiation source 11 with a subject M interposed therebetween.

A reading controller 14 is connected with the imaging console 2. The reading controller 14 controls the switches of the pixels of the radiation detector 13 on the basis of image reading conditions input from the imaging console 2 to switch the pixels to read the electric signals accumulated in the pixels, thereby reading the electric signals accumulated in the radiation detector 13 and obtaining image data. This image data is a frame image(s). The reading controller 14 outputs the obtained frame images to the imaging console 2. The image reading conditions include a frame rate, a frame interval, a pixel size, and an image size (matrix size). The frame rate is the number of frame images to be obtained per second, and matches the pulse rate described above. The frame interval is a period of time from the start of one frame image obtaining action to the start of the next frame image obtaining action, and matches the pulse interval described above.

The radiation emission controller 12 and the reading controller 14 are connected to each other, and exchange sync signals so as to synchronize radiation emission actions with image reading actions.

### [Configuration of Imaging Console 2]

The imaging console 2 outputs the radiation emission conditions and the image reading conditions to the imager 1 so as to control the radiation imaging and the radiation image reading actions performed by the imager 1, and also displays the dynamic image obtained by the imager 1 so that a radiographer, such as a radiologist, can check if positioning has no problem, and also can determine if the dynamic image is suitable for diagnosis.

The imaging console 2 includes, as shown in FIG. 1, a controller 21, a storage 22, an operation unit 23, a display 24 and a communication unit 25. These units or the like are connected to one another via a bus 26.

The controller 21 includes a CPU (Central Processing Unit) and a RAM (Random Access Memory). The CPU of the controller 21 reads a system program and various process programs stored in the storage 22 in response to operations on the operation unit 23, opens the read programs in the RAM, and performs various processes, such as the below-described imaging control process, in accordance with the opened programs, thereby performing concentrated control of actions of the units or the like of the imaging console 2 and the radiation emission actions and the reading actions of the imager 1.

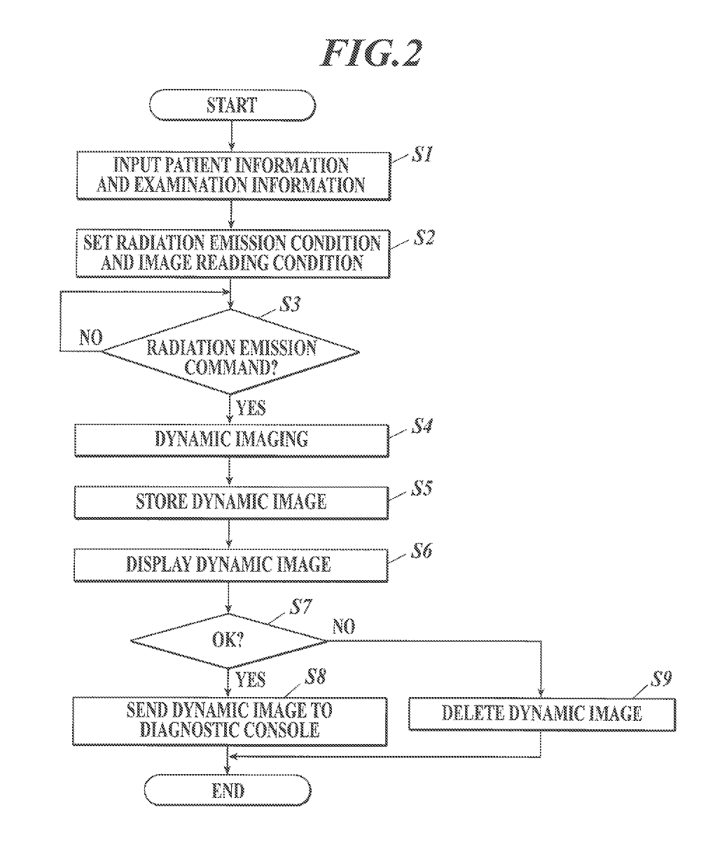

The storage 22 is constituted of a nonvolatile semiconductor memory, a hard disk or the like. The storage 22 stores therein various programs to be executed by the controller 21, parameters necessary to perform processes of the programs, data, such as process results, and so forth. For example, the storage 22 stores therein a program for the imaging control process shown in FIG. 2. The storage 22 also stores therein the radiation emission conditions and the image reading conditions for respective imaging sites. The programs are stored in the form of a computer readable program code(s), and the controller 21 acts in accordance with the program code.

The operation unit 23 includes: a keyboard including cursor keys, number input keys and various function keys; and a pointing device, such as a mouse, and outputs, to the controller 21, command signals input by key operations on the keyboard or by mouse operations. The operation unit 23 may have a touchscreen on the display screen of the display 24. In this case, the operation unit 23 outputs command signals input via the touchscreen to the controller 21.

The display 24 is constituted of a monitor, such as an LCD (Liquid Crystal Display) or a CRT (Cathode Ray Tube), and displays thereon commands input from the operation unit 23, data and so forth in accordance with commands of display signals input from the controller 21.

The communication unit 25 includes a LAN adapter, a modem and a TA (Terminal Adapter), and controls data exchange with apparatuses connected to the communication network NT.

### [Configuration of Diagnostic Console 3]

The diagnostic control 3 is a dynamic image processing apparatus that obtains the dynamic image from the imaging console 2, performs image processing and/or analysis on the obtained dynamic image, and displays the obtained dynamic image and/or the analysis result to help a doctor(s) make a diagnosis. The diagnostic console 3 includes, as shown in FIG. 1, a controller 31, a storage 32, an operation unit 33, a display 34 and a communication unit 35. These units or the like are connected to one another via a bus 36.

The controller 31 includes a CPU (hardware processor) and a RAM. The CPU of the controller 31 reads a system program and various process programs stored in the storage 32 in response to operations on the operation unit 33, opens the read programs in the RAM, and performs various processes, such as the below-described frequency emphasis process A, in accordance with the opened programs.

The storage 32 is constituted of a nonvolatile semiconductor memory, a hard disk or the like. The storage 32 stores therein various programs, including a program for the frequency emphasis process A, to be executed by the controller 31, parameters necessary to perform processes of the programs, data, such as process results, and so forth. The programs are stored in the form of a computer readable program code(s), and the controller 31 acts in accordance with the program code.

The operation unit 33 includes: a keyboard including cursor keys, number input keys and various function keys; and a pointing device, such as a mouse, and outputs, to the controller 31, command signals input by key operations on the keyboard or by mouse operations. The operation unit 33 may have a touchscreen on the display screen of the display 34. In this case, the operation unit 33 outputs command signals input via the touchscreen to the controller 31.

The display 34 is constituted of a monitor, such as an LCD or a CRT, and performs various types of display in accordance with commands of display signals input from the controller 31.

The communication unit 35 includes a LAN adapter, a modem and a TA, and controls data exchange with apparatuses connected to the communication network NT.

### [Actions of Dynamic Image Processing System 100]

Next, actions of the dynamic image processing system 100 are described.

### [Actions of Imager 1 and Imaging Console 2]

First, imaging actions performed by the imager 1 and the imaging console 2 are described.

FIG. 2 shows the imaging control process performed by the controller 21 of the imaging console 2. The imaging control process is performed by the controller 21 in cooperation with the program stored in the storage 22.

First, a radiographer operates the operation unit 23 of the imaging console 2 so as to input patient information (patient name, height, weight, age, sex, etc.) on an examinee, and examination information (an imaging site (here, the chest), a type of diagnosis target (ventilation or lung perfusion (hereinafter may be simply referred to as “perfusion”)), etc.) on an examination to be performed on the examinee (Step S1).

Next, the controller 21 reads radiation emission conditions from the storage 22 so as to set them in the radiation emission controller 12, and also reads image reading conditions from the storage 22 so as to set them in the reading controller 14 (Step S2).

Next, the controller 21 waits for a radiation emission command to be input by the radiographer operating the operation unit 23 (Step S3). Here, the radiographer places a subject M between the radiation source 11 and the radiation detector 13 and performs positioning. Further, the radiographer instructs the examinee to relax and encourages him/her to do quiet breathing, or may lead the examinee to deep breathing by saying “Breathe in.”, “Breathe out.” and so forth. When preparations for imaging are complete, the radiographer operates the operation unit 23 so as to input the radiation emission command

When receiving the radiation emission command input through the operation unit 23 (Step S3; YES), the controller 21 outputs an imaging start command to the radiation emission controller 12 and the reading controller 14 to start dynamic imaging (Step S4). That is, the radiation source 11 emits radiation at pulse intervals set in the radiation emission controller 12, and accordingly the radiation detector 13 obtains (generates) a series of frame images.

When imaging for a predetermined number of frame images finishes, the controller 21 outputs an imaging end command to the radiation emission controller 12 and the reading controller 14 to stop the imaging actions. The number of frame images to be taken covers at least one breathing cycle or pulsation cycle.

The frame images obtained by imaging are successively input to the imaging console 2 and stored in the storage 22, the frame images being correlated with respective numbers indicating what number in the imaging order the respective frame images have been taken (frame numbers) (Step S5), and also displayed on the display 24 (Step S6). The radiographer checks the positioning or the like with the displayed dynamic image, and determines whether the dynamic image obtained by dynamic imaging is suitable for diagnosis (Imaging OK) or re-imaging is necessary (Imaging NG). Then, the radiographer operates the operation unit 23 so as to input the determination result.

When the determination result “Imaging OK” is input by the radiographer performing a predetermined operation on the operation unit 23 (Step S7; YES), the controller 21 attaches, to the respective frame images obtained by dynamic imaging (e.g. writes, in the header region of the image data in DICOM), information such as an ID to identify the dynamic image, the patient information, the examination information, the radiation emission conditions, the image reading conditions, and the respective numbers indicating what number in the imaging order the respective frame images have been taken (frame numbers), and sends the same to the diagnostic console 3 through the communication unit 25 (Step S8), and then ends the imaging control process. On the other hand, when the determination result “Imaging NG” is input by the radiographer performing a predetermined operation on the operation unit 23 (Step S7; NO), the controller 21 deletes the frame images (the series of frame images) from the storage 22 (Step S9), and then ends the imaging control process. In this case, re-imaging is necessary.

### [Actions of Diagnostic Console 3]

Next, actions of the diagnostic console 3 are described.

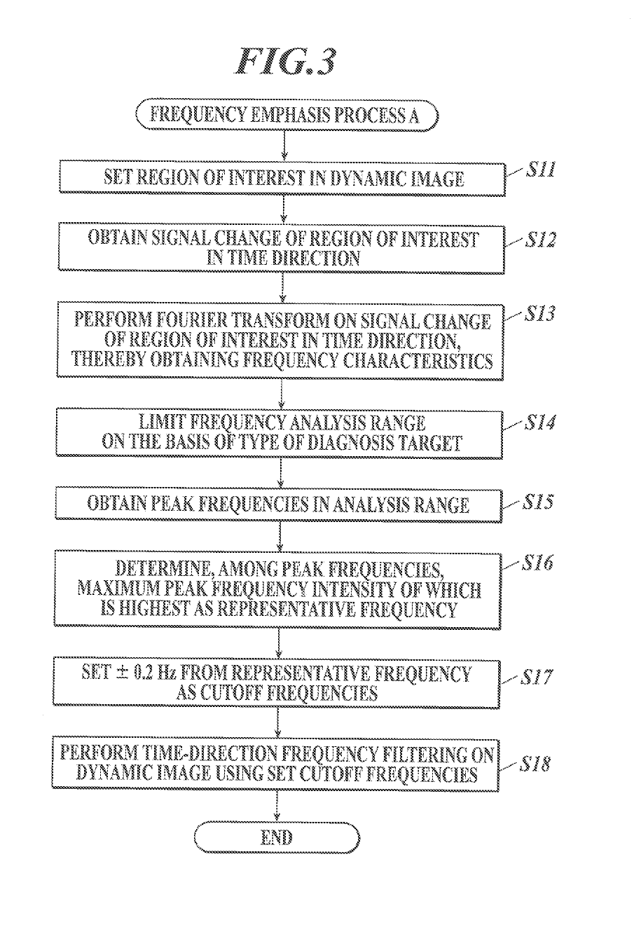

In the diagnostic console 3, when receiving a series of frame images of a dynamic image from the imaging console 2 through the communication unit 35, the controller 31 performs the frequency emphasis process A shown in FIG. 3 in cooperation with the program stored in the storage 32.

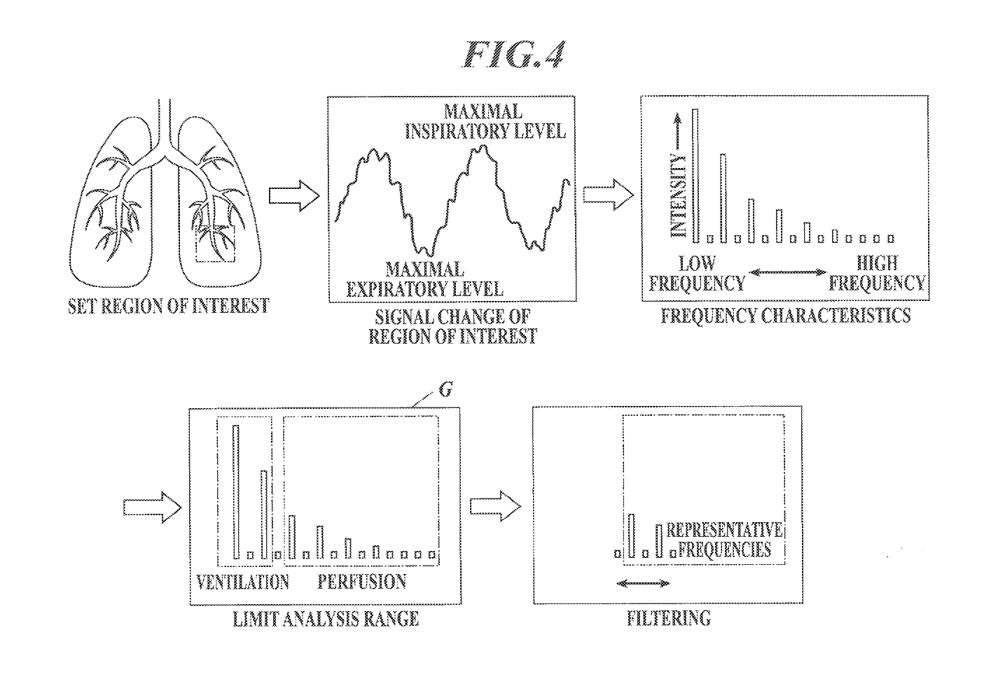

Hereinafter, the flow of the frequency emphasis process A is described with reference to FIG. 3 and FIG. 4.

First, the controller 31 sets a region of interest in a received dynamic image (Step S11).

In Step S11, the region of interest may be set automatically or may be set in response to a user operation, namely, may be set manually by a user setting it on the dynamic image displayed on the display 34 by operating the operation unit 33. The shape and the number of regions of interest to be set are not particularly limited. The region of interest is set preferably on the trajectory of the diaphragm if the type of dynamic target is ventilation or on the region of the heart if the type of dynamic target is lung perfusion. Setting the region of interest, which is used to calculate cutoff frequencies, at a position where the signal component of the dynamic state to be diagnosed (diagnosis target) is dominant enables appropriate extraction of the signal component of the dynamic state to be diagnosed.

If the region of interest is set automatically, in the case where the type of diagnosis target is ventilation, for example, a lung region(s) is extracted from each frame image of the dynamic image, the contour of the bottom part of the extracted lung region is recognized as the diaphragm, and a rectangle or square containing the upper limit and the lower limit of the trajectory of the diaphragm (the upper end and the lower end of a diaphragm movement range) is set as the region of interest. Any method can be used for extraction of the lung region. For example, a threshold value is obtained from a histogram of signal values of pixels of a frame image by discriminant analysis, and a region having a higher signal value(s) than the threshold value is extracted as a lung region candidate. Then, edge detection is performed on around the border of the extracted lung region candidate, and, in small regions around the border, points where the edge is the maximum are extracted along the border. Thus, the border of the lung region can be extracted.

In the case where the type of diagnosis target is lung perfusion, for example, the contour of the heart is extracted from each frame image of the dynamic image, and the region of interest is set on a region inside the extracted contour of the heart. Extraction of the contour of the heart can be performed by a well-known image processing technique, such as a method for determining the contour of a heart described in Japanese Patent No. 2796381.

Next, the controller 31 obtains signal change of the region of interest in the time direction (Step S12). For example, the controller 31 calculates, for each frame image, a representative value (e.g. the mean value, the maximum value, the minimum value, etc.) of signal values (density values) of pixels in the region of interest, and obtains change of the calculated representative value in the time direction as signal change of the region of interest in the time direction (Step S12). Obtaining not time change of a signal value per pixel but time change of a representative value of signal values of pixels in the region of interest can reduce noise.

Next, the controller 31 performs Fourier transform on the signal change of the region of interest in the time direction, thereby obtaining frequency characteristic(s) (intensity of each frequency) of the signal change of the region of interest in the time direction (Step S13). Walsh transform and Wavelet transform can be used as the method for obtaining the frequency characteristics.

Next, the controller 31 limits the range of frequencies to be analyzed (analysis range) on the basis of the type of diagnosis target (Step S14), and obtains peak frequencies in the analysis range (Step S15). The peak frequencies are each a frequency the intensity of which is larger than intensities of its neighbors. If the type of diagnosis target is ventilation, the analysis range is limited to the low frequency side of 0.8 Hz (i.e. lower than 0.8 Hz), whereas if the type of diagnosis target is lung perfusion, the analysis range is limited to 0.8 Hz and the high frequency side thereof (i.e. 0.8 Hz or higher).

Next, the controller 31 determines, among the peak frequencies in the analysis range, the maximum peak frequency the intensity of which is highest as a representative frequency (Step S16), and sets ±0.2 Hz from the representative frequency as cutoff frequencies (Step S17). That is, cutoff frequencies to emphasize only the fundamental frequency of the dynamic state to be diagnosed are set. The “±0.2 Hz” is value(s) determined on the basis of frequency resolution, but this is not a limit.

As shown in the graph G in FIG. 4, a dynamic image obtained by X-ray imaging of a chest contains the low-frequency signal component(s) due to ventilation and the high-frequency signal component(s) due to perfusion. Hence, for example, if cutoff frequencies are set on the basis of the center frequency or the mean frequency of frequencies obtained from the dynamic image, the maximum peak frequency, namely, the fundamental frequency, of the dynamic state to be diagnosed (ventilation or lung perfusion) may not be emphasized. Hence, in the embodiments (first to fourth embodiments), one or more representative frequencies are determined in such a way as to include the maximum peak frequency in the frequency range (analysis range) limited on the basis of the type of diagnosis target, and cutoff frequencies are set such that the maximum peak frequency of the dynamic state to be diagnosed is contained in a frequency range that is emphasized (extracted), without exception.

Then, the controller 31 performs the time-direction frequency filtering on the dynamic image using a bandpass filter having the set cutoff frequencies (Step S18), and then ends the frequency emphasis process A.

The controller 31 causes the display 34 to display the dynamic image, which has been subjected to the frequency filtering, or analyzes the dynamic image and causes the display 34 to display the analysis result.

As described above, in the frequency emphasis process A, the controller 31 obtains, as a representative frequency, the maximum peak frequency in the frequency range (analysis range) for the type of diagnosis target on the basis of the obtained dynamic image, sets cutoff frequencies in the vicinity of the obtained maximum peak frequency on the low frequency side and the high frequency side of the maximum peak frequency as a reference, and performs the time-direction frequency filtering on the dynamic image using the set cutoff frequencies. This can emphasize only the fundamental frequency of the dynamic state to be diagnosed of a patient (examinee) with high accuracy and hence can generate a dynamic image having a high signal-to-noise ratio, and accordingly can enhance readability of the information on the dynamic state to be diagnosed.

### Second Embodiment

Hereinafter, a second embodiment is described.

Configuration (components) of the second embodiment is the same as that of the first embodiment. Hence, descriptions thereof are not repeated here.

Further, configurations (components) of the imager 1, the imaging console 2 and the diagnostic console 3 and actions of the imager 1 and the imaging console 2 in the second embodiment are the same as those in the first embodiment. Hence, descriptions thereof are not repeated here, and actions of the diagnostic console 3 are described.

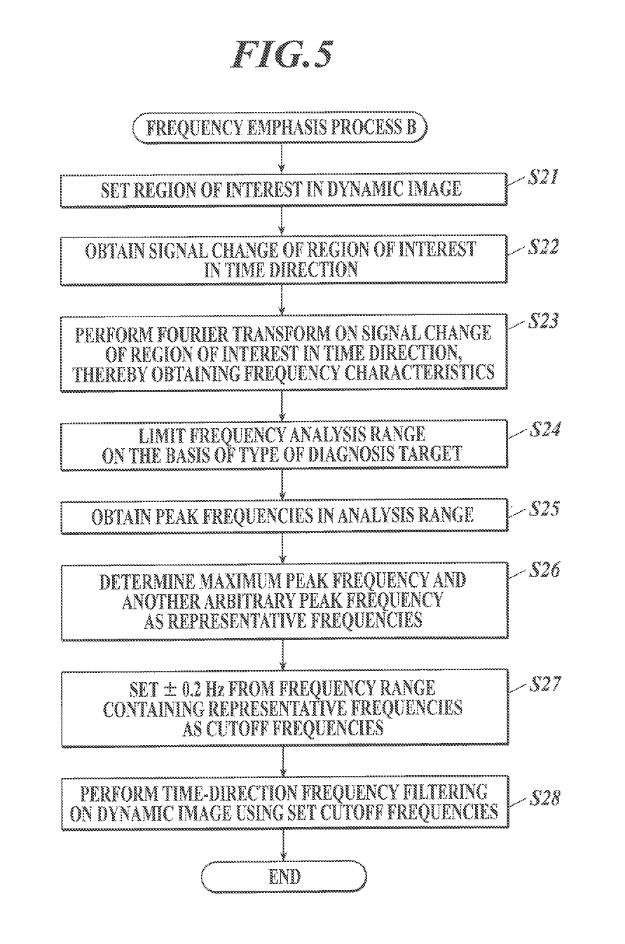

In the diagnostic console 3, when receiving a series of frame images of a dynamic image from the imaging console 2 through the communication unit 35, the controller 31 performs a frequency emphasis process B shown in FIG. 5 in cooperation with the program stored in the storage 32.

Hereinafter, the flow of the frequency emphasis process B is described with reference to FIG. 5.

First, the controller 31 obtains peak frequencies in the analysis range by performing Steps S21 to S25. Steps S21 to S25 are the same as Steps S11 to S15 in FIG. 3 described in the first embodiment. Hence, descriptions thereof are not repeated here.

Next, the controller 31 determines, among the peak frequencies in the analysis range, the maximum peak frequency the intensity of which is highest and an arbitrary peak frequency (one or more arbitrary peak frequencies) other than the maximum peak frequency as representative frequencies (Step S26), and sets ±0.2 Hz from a frequency range containing the representative frequencies as cutoff frequencies (Step S27). The arbitrary peak frequency can be set in advance with the operation unit 33. The number of arbitrary peak frequencies is not particularly limited. The frequency range containing the representative frequencies is a range from the minimum frequency to the maximum frequency among the representative frequencies. The “±0.2 Hz” is a value(s) determined on the basis of frequency resolution, but this is not a limit.

That is, in Step S27, cutoff frequencies to emphasize the frequency range containing the fundamental frequency and its harmonic(s) of the dynamic state to be diagnosed are set. The frequency range containing not only the fundamental frequency but also its harmonic(s) can adjust the waveform of the signal component to be contained in a dynamic image to a shape corresponding to the waveform of the actual dynamic state.

Then, the controller 31 performs the time-direction frequency filtering on the dynamic image using a bandpass filter having the set cutoff frequencies (Step S28), and then ends the frequency emphasis process B.

The controller 31 causes the display 34 to display the dynamic image, which has been subjected to the frequency filtering, or analyzes the dynamic image and causes the display 34 to display the analysis result.

As described above, in the frequency emphasis process B, the controller 31 obtains, as representative frequencies, the maximum peak frequency and at least one arbitrary peak frequency (harmonic of the maximum peak frequency) in the frequency range (analysis range) for the type of diagnosis target on the basis of the obtained dynamic image, sets cutoff frequencies on the low frequency side and the high frequency side of a frequency range containing the obtained representative frequencies, and performs the time-direction frequency filtering on the dynamic image using the set cutoff frequencies. This can emphasize only the frequency range containing the fundamental frequency and its harmonic(s) of the dynamic state to be diagnosed of a patient (examinee) and hence can generate a dynamic image which keeps a sufficient signal-to-noise ratio and in which the signal waveform of the dynamic state to be diagnosed has an appropriate shape, and accordingly can enhance readability of the information on the dynamic state to be diagnosed.

### Third Embodiment

Hereinafter, a third embodiment is described.

Configuration (components) of the third embodiment is the same as that of the first embodiment. Hence, descriptions thereof are not repeated here.

Further, configurations (components) of the imager 1, the imaging console 2 and the diagnostic console 3 and actions of the imager 1 and the imaging console 2 in the third embodiment are the same as those in the first embodiment. Hence, descriptions thereof are not repeated here, and actions of the diagnostic console 3 are described.

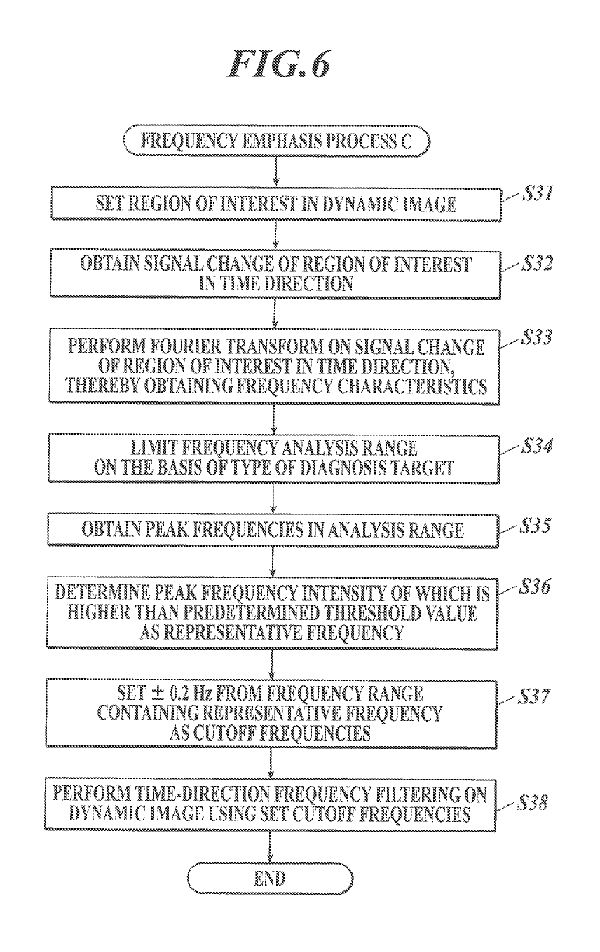

In the diagnostic console 3, when receiving a series of frame images of a dynamic image from the imaging console 2 through the communication unit 35, the controller 31 performs a frequency emphasis process C shown in FIG. 6 in cooperation with the program stored in the storage 32.

Hereinafter, the flow of the frequency emphasis process C is described with reference to FIG. 6.

First, the controller 31 obtains peak frequencies in the analysis range by performing Steps S31 to S35. Steps S31 to S35 are the same as Steps S11 to S15 in FIG. 3 described in the first embodiment. Hence, descriptions thereof are not repeated here.

Next, the controller 31 determines, among the peak frequencies in the analysis range, a peak frequency (or peak frequencies) the intensity of which is higher than a predetermined threshold value as a representative frequency (or representative frequencies) (Step S36), and sets ±0.2 Hz from a frequency range containing the representative frequency as cutoff frequencies (Step S37). The threshold value for intensities can be set in advance with the operation unit 33. This threshold value for intensities is a value with which at least one peak frequency is determined as a representative frequency. The “±0.2 Hz” is a value(s) determined on the basis of frequency resolution, but this is not a limit.

That is, in Step S37, cutoff frequencies to emphasize the fundamental frequency of the dynamic state to be diagnosed or the fundamental frequency and its harmonic(s) thereof are set.

Then, the controller 31 performs the time-direction frequency filtering on the dynamic image using a bandpass filter having the set cutoff frequencies (Step S38), and then ends the frequency emphasis process C.

The controller 31 causes the display 34 to display the dynamic image, which has been subjected to the frequency filtering, or analyzes the dynamic image and causes the display 34 to display the analysis result.

As described above, in the frequency emphasis process C, the controller 31 obtains, as a representative frequency (or representative frequencies), at least one peak frequency the intensity of which is higher than a predetermined threshold value in the frequency range (analysis range) for the type of diagnosis target on the basis of the obtained dynamic image, sets cutoff frequencies on the low frequency side and the high frequency side of a frequency range containing the obtained representative frequency, and performs the time-direction frequency filtering on the dynamic image using the set cutoff frequencies. This can emphasize the fundamental frequency (or the fundamental frequency and its harmonic(s)) of the dynamic state to be diagnosed of a patient (examinee). That is, this can emphasize only the fundamental frequency of the dynamic state to be diagnosed of a patient (examinee) with high accuracy and hence can generate a dynamic image having a high signal-to-noise ratio, and accordingly can enhance readability of the dynamic state to be diagnosed. Alternatively, this can generate a dynamic image which keeps a sufficient signal-to-noise ratio and in which the signal waveform of the dynamic state to be diagnosed has an appropriate shape.

### Fourth Embodiment

Hereinafter, a fourth embodiment is described.

Configuration (components) of the fourth embodiment is the same as that of the first embodiment. Hence, descriptions thereof are not repeated here.

Further, configurations (components) of the imager 1, the imaging console 2 and the diagnostic console 3 and actions of the imager 1 and the imaging console 2 in the fourth embodiment are the same as those in the first embodiment. Hence, descriptions thereof are not repeated here, and actions of the diagnostic console 3 are described.

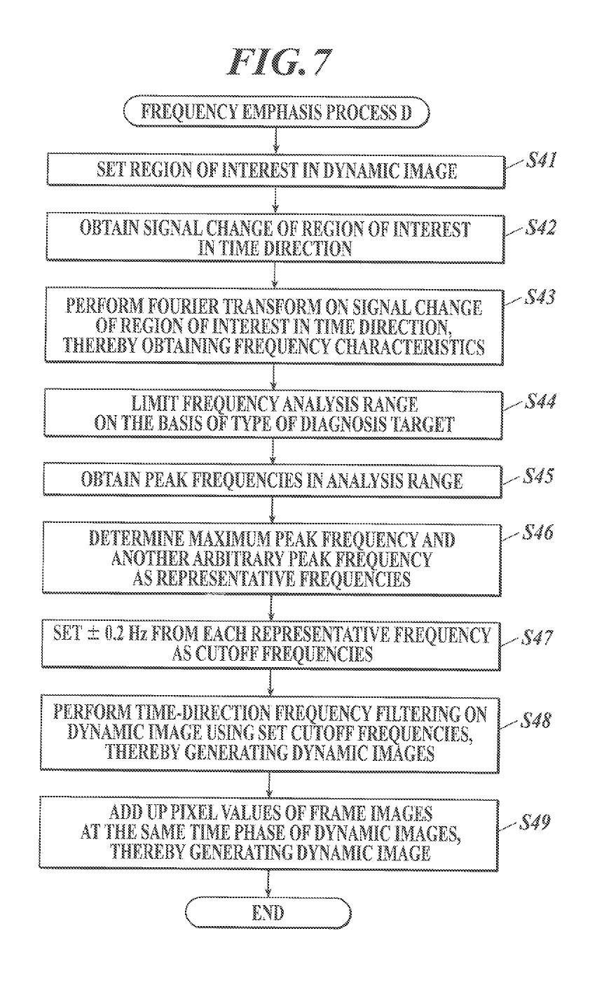

In the diagnostic console 3, when receiving a series of frame images of a dynamic image from the imaging console 2 through the communication unit 35, the controller 31 performs a frequency emphasis process D shown in FIG. 7 in cooperation with the program stored in the storage 32.

Hereinafter, the flow of the frequency emphasis process D is described with reference to FIG. 7.

First, the controller 31 obtains peak frequencies in the analysis range by performing Steps S41 to S45. Steps S41 to S45 are the same as Steps S11 to S15 in FIG. 3 described in the first embodiment. Hence, descriptions thereof are not repeated here.

Next, the controller 31 determines, among the peak frequencies in the analysis range, the maximum peak frequency the intensity of which is highest and an arbitrary peak frequency (one or more arbitrary peak frequencies) other than the maximum peak frequency as representative frequencies (Step S46), and sets ±0.2 Hz from each representative frequency as cutoff frequencies (Step S47). The arbitrary peak frequency can be set in advance with the operation unit 33. The number of arbitrary peak frequencies is not particularly limited. The “±0.2 Hz” is a value(s) determined on the basis of frequency resolution, but this is not a limit.

That is, in Step S47, cutoff frequencies to emphasize the fundamental frequency of the dynamic state to be diagnosed and cutoff frequencies to emphasize its harmonic(s) are set.

Then, the controller 31 performs the time-direction frequency filtering on the dynamic image using each bandpass filter having the set cutoff frequencies, thereby generating a plurality of dynamic images (Step S48). That is, the controller 31 performs the time-direction frequency filtering on the dynamic image using the set cutoff frequencies the number of times that is the same as the number of the representative frequencies, thereby generating a plurality of dynamic images.

Then, the controller 31 adds up pixel values of frame images at the same time phase of the dynamic images generated by the frequency filtering, thereby generating a dynamic image (Step S49), and then ends the frequency emphasis process D.

The controller 31 causes the display 34 to display the generated dynamic image, or analyzes the dynamic image and causes the display 34 to display the analysis result.

As described above, in the frequency emphasis process D, the controller 31 obtains, as representative frequencies, the maximum peak frequency and at least one arbitrary peak frequency (harmonic of the maximum peak frequency) in the frequency range (analysis range) for the type of diagnosis target on the basis of the obtained dynamic image, sets cutoff frequencies on the low frequency side and the high frequency side of each of the obtained representative frequencies, and performs the time-direction frequency filtering on the dynamic image using each pair of the cutoff frequencies, thereby generating a plurality of dynamic images. Then, the controller 31 adds up pixel values of frame images at the same time phase of the dynamic images, thereby generating a dynamic image. This can emphasize only the fundamental frequency and its harmonic(s) of the dynamic state to be diagnosed of a patient (examinee) and hence can generate a dynamic image which has a high signal-to-noise ratio and in which the signal waveform of the dynamic state to be diagnosed has an appropriate shape, and accordingly can enhance readability of the information on the dynamic state to be diagnosed.

As described above, according to the diagnostic console 3, the controller 31: obtains frequency characteristic(s) of density change of a dynamic image in the time direction by Fourier transform, the dynamic image being obtained by imaging at least one breathing cycle or pulsation cycle; obtains peak frequencies in a frequency range for a type of diagnosis target; determines at least one representative frequency including the maximum peak frequency the intensity of which is highest among the peak frequencies in the frequency range for the type of diagnosis target; and performs the time-direction filtering on the dynamic image using cutoff frequencies to emphasize (extract) the determined representative frequency.

Thus, the controller 31 performs the time-direction filtering on the dynamic image using the cutoff frequencies to emphasize at least one representative frequency including the maximum peak frequency among the peak frequencies in the frequency range for the type of diagnosis target. This can emphasize the maximum peak frequency of the dynamic state to be diagnosed without exception, and accordingly can enhance readability of the information on the dynamic state to be diagnosed.

For example, the controller 31: determines the maximum peak frequency as a representative frequency; sets cutoff frequencies on the low frequency side and the high frequency side of the determined representative frequency as a reference, respectively; and performs the time-direction filtering on the dynamic image using the set cutoff frequencies. This can emphasize only the fundamental frequency of the dynamic state to be diagnosed of a patient (examinee) with high accuracy and hence can generate a digenic image having a high signal-to-noise ratio, and accordingly can enhance readability of the information on the dynamic state to be diagnosed.

Alternatively, for example, the controller 31: determines, among the obtained peak frequencies, the maximum peak frequency and at least one peak frequency the intensity of which is lower than the intensity of the maximum peak frequency as representative frequencies; sets cutoff frequencies on the low frequency side and the high frequency side of a frequency range containing the determined representative frequencies, respectively; and performs the time-direction filtering on the dynamic image using the set cutoff frequencies. This can emphasize only the frequency range containing the fundamental frequency and its harmonic(s) of the dynamic state to be diagnosed of a patient (examinee) and hence can generate a diagnosis image which keeps a sufficient signal-to-noise ratio and in which the signal waveform of the dynamic state to be diagnosed has an appropriate shape, and accordingly can enhance readability of the information on the dynamic state to be diagnosed.

Alternatively, for example, the controller 31: determines, among the obtained peak frequencies, at least one peak frequency the intensity of which is higher than a predetermined threshold value as at least one representative frequency; sets cutoff frequencies on the low frequency side and the high frequency side of a frequency range containing the determined representative frequency, respectively; and performs the time-direction filtering on the dynamic image using the set cutoff frequencies. This can emphasize only the fundamental frequency (or the fundamental frequency and its harmonic(s)) of the dynamic state to be diagnosed of a patient (examinee) and hence can generate a dynamic image having a high signal-to-noise ratio, and accordingly can enhance readability of the dynamic state to be diagnosed. Alternatively, this can generate a dynamic image which keeps a sufficient signal-to-noise ratio and in which the signal waveform of the dynamic state to be diagnosed has an appropriate shape.

Alternatively, for example, the controller 31: determines, among the obtained peak frequencies, the maximum peak frequency and at least one peak frequency the intensity of which is lower than the intensity of the maximum peak frequency as representative frequencies; sets cutoff frequencies on the low frequency side and the high frequency side of each of the representative frequencies, respectively; performs the time-direction filtering on the dynamic image using the set cutoff frequencies the number of times that is the same as the number of the representative frequencies, thereby generating a plurality of dynamic images; and adds up pixel values of frame images at the same time phase of the generated dynamic images, thereby generating a dynamic image. This can emphasize only the fundamental frequency and its harmonic(s) of the dynamic state to be diagnosed of a patient (examinee) and hence can generate a dynamic image which has a high signal-to-noise ratio and in which the signal waveform of the dynamic state to be diagnosed has an appropriate shape, and accordingly can enhance readability of the information on the dynamic state to be diagnosed.

Those described in the above embodiments are preferred examples of the dynamic image processing system of the present invention, and not intended to limit the present invention.

For example, in the above embodiments, the controller 31 limits the range of frequencies to be analyzed (analysis range) according to the dynamic state to be diagnosed, obtains peak frequencies in the limited analysis range, and determines at least one representative frequency. Alternatively, the controller 31 may obtain peak frequencies of the dynamic image, obtain only peak frequencies in the frequency range (analysis range) for the type of diagnosis target, and determine at least one representative frequency.

Further, for example, in the above, as a computer readable medium for the programs of the present invention, a hard disk, a nonvolatile semiconductor memory or the like is used. However, this is not a limit. As the computer readable medium, a portable recording/storage medium, such as a CD-ROM, can also be used. Further, as a medium to provide data of the programs of the present invention, a carrier wave can be used.

In addition to the above, the specific configurations/components and the specific actions of the apparatuses of the dynamic image processing system 100 can also be appropriately modified without departing from the spirit of the present invention.

Although embodiments of the present invention have been described and illustrated in detail, it is clearly understood that the same is by way of illustration and example only and is not to be taken by way of limitation, and the scope of the present invention should be interpreted by terms of the appended claims

What is claimed is:

1. A dynamic image processing apparatus comprising a hardware processor that:
obtains a frequency characteristic of density change of a dynamic image in a time direction, the dynamic image being obtained by imaging at least one breathing cycle or pulsation cycle;
obtains peak frequencies in a frequency range for a type of diagnosis target based on the obtained frequency characteristic;
determines at least one representative frequency including a maximum peak frequency an intensity of which is highest among the obtained peak frequencies; and
performs time-direction filtering on the dynamic image using cutoff frequencies to emphasize the determined representative frequency.

2. The dynamic image processing apparatus according to claim 1, wherein the hardware processor:
sets a region of interest on a trajectory of a diaphragm in the dynamic image if the type of diagnosis target is ventilation or on a region of a heart in the dynamic image if the type of diagnosis target is lung perfusion; and
obtains the frequency characteristic of the density change of the set region of interest in the time direction.

3. The dynamic image processing apparatus according to claim 2, wherein the hardware processor:
calculates, for each frame image of the dynamic image, a representative value of density values of pixels in the region of interest; and
obtains time change of the calculated representative value as the density change of the dynamic image in the time direction.

4. The dynamic image processing apparatus according to claim 1, wherein the hardware processor obtains the peak frequencies in the frequency range limited for the type of diagnosis target based on a predetermined threshold value.

5. The dynamic image processing apparatus according to claim 1, wherein the hardware processor:
determines the maximum peak frequency as the representative frequency;
sets the cutoff frequencies on a low frequency side and a high frequency side of the determined representative frequency as a reference, respectively; and
performs the time-direction filtering on the dynamic image using the set cutoff frequencies.

6. The dynamic image processing apparatus according to claim 1, wherein the hardware processor:
determines, among the obtained peak frequencies, the maximum peak frequency and at least one peak frequency an intensity of which is lower than the intensity of the maximum peak frequency as representative frequencies, which are the at least one representative frequency;
sets the cutoff frequencies on a low frequency side and a high frequency side of a frequency range containing the determined representative frequencies, respectively; and
performs the time-direction filtering on the dynamic image using the set cutoff frequencies.

7. The dynamic image processing apparatus according to claim 1, wherein the hardware processor:
determines, among the obtained peak frequencies, at least one peak frequency an intensity of which is higher than a predetermined threshold value as the at least one representative frequency;
sets the cutoff frequencies on a low frequency side and a high frequency side of a frequency range containing the determined representative frequency, respectively; and
performs the time-direction filtering on the dynamic image using the set cutoff frequencies.

8. The dynamic image processing apparatus according to claim 1, wherein the hardware processor:
determines, among the obtained peak frequencies, the maximum peak frequency and at least one peak frequency an intensity of which is lower than the intensity of the maximum peak frequency as representative frequencies, which are the at least one representative frequency;
sets the cutoff frequencies on a low frequency side and a high frequency side of each of the representative frequencies, respectively;
performs the time-direction filtering on the dynamic image using the set cutoff frequencies a number of times that is same as a number of the representative frequencies, thereby generating a plurality of dynamic images; and
adds up pixel values of frame images at a same time phase of the generated dynamic images, thereby generating a dynamic image.
